(19) United States
(12) Patent Application Publication
(10) Pub. No.: US 2021/0100670 A1
(43) Pub. Date: Apr. 8, 2021

(54) BEVELED CORONARY STENT AND CATHETER FOR USE IN A BRANCH WITH LESS THAN 90º ANGLE AND ASSOCIATED METHODS

(71) Applicant: Hani Seifein, Maitland, FL (US)

(72) Inventor: Hani Seifein, Maitland, FL (US)

(21) Appl. No.: 16/596,245

(22) Filed: Oct. 8, 2019

(51) Int. Cl. A61F 2/954; A61M 25/00; A61M 25/10; A61F 2/90; A61F 2/958

(52) U.S. Cl. CPC A61F 2/954; A61M 25/0026; A61M 25/10; A61F 2/90; A61F 2/958; A61F 2230/0017; A61M 2025/1079; A61M 2025/0037; A61M 2025/0008; A61M 2025/1045; A61M 2025/1061; A61F 2250/0098

(57) ABSTRACT

The system is for treating a bifurcated vessel in a patient. The system includes a catheter, a primary expandable stent delivery balloon, and a radially expandable beveled stent with a bevel angle at a leading edge. The primary expandable stent delivery balloon is configured to transverse the ostium and protrude into the main vessel from the branch vessel during placement of the beveled stent. The primary expandable stent delivery balloon includes three-dimensional (3-D) orientation markers including a triangular shaped proximal radiopaque marker configured to demarcate a triangle defined by a difference between the longer side and the shorter side at the leading edge at the beveled proximal end of the beveled stent, and a distal radiopaque marker that is positioned at the distal portion of the primary expandable stent delivery balloon and configured to identify, at the distal end of the beveled stent, the longer side of the beveled stent.

### FIELD OF THE INVENTION

The present invention relates to systems and methods for coronary intervention with the use of stents and catheters.

### BACKGROUND OF THE INVENTION

Coronary heart disease is treated with percutaneous coronary intervention (PCI) methods. Thereby, a flow restriction in a vessel is removed by dilating the blocked vessel with an inflatable balloon, for example. PCI can lead to a recoil, collapse or restenosis of the vessel in the treated area. Therefore, after or during dilating the vessel which could be an artery or a vein, a stent may be implanted into the treated vessel to keep it open and/or to reinforce it.

Typically, a stent is a metallic mesh in the form of a hollow cylinder. The stent is introduced into the body in an unexpanded state and is expanded at the site where the stent should be placed to its final diameter. The stent thus acts as a scaffold that reinforces the wall of the vessel. Vessels may branch or bifurcate like arteries or flow together like veins. Typically, the angulations between a parent vessel and a side branch that leads into the parent vessel range widely, e.g. in human coronary arteries between about 30° to about 120°. Vascular lesions are frequently located at this branching or bifurcation point.

Conventional stents have been used to treat more complex vascular problems, such as lesions at or near the bifurcation points in the vascular system. A bifurcation is where a secondary artery (sometimes referred to as a branch vessel) branches out of a typically larger vessel (sometimes referred to as the main vessel). Stenting of bifurcations can present may challenges. For example, a stent that traverses the ostium of the branch vessel may obstruct blood flow into the branch. Moreover, the struts in a stent may also block the branch vessel, limiting or preventing access to the branch by another diagnostic or therapeutic device such as another catheter.

FIG. 1 is a longitudinal cross-sectional view of a bifurcated main vessel with a conventional non-beveled stent placed at a 90-degree branch vessel. FIGS. 2A and 2B are longitudinal cross-sectional views of a bifurcated main vessel with a conventional non-beveled stent placed at a less-than-90-degree branch vessel. In FIG. 2A the stent placement misses a triangular portion M of the branch vessel at the ostium. In FIG. 2B the stent placement protrudes P into the main vessel.

U.S. Published Patent Application No. 2016/0100966 to Bourang and entitled ‘Multi-Stent and Multi-Balloon Apparatus for Treating Bifurcations and Methods of Use” is directed to a system for treating a bifurcated vessel that includes a first delivery catheter and a second delivery catheter. The first delivery catheter carries a proximal first stent and a distal second stent. The first delivery catheter also has a first elongate shaft, a proximal first expandable member with the proximal first stent disposed thereover, and a distal second expandable member with the distal second stent disposed thereover. The proximal first expandable member and distal second expandable member are independently expandable of one another. The second delivery catheter carries a third stent. The second delivery catheter also has a second elongate shaft, and a third expandable member with the third stent disposed thereover. The third expandable member is independently expandable of the proximal first expandable member and the distal second expandable member.

U.S. Published Patent Application No. 2009/0005857 to Ischinger and entitled “Lesion Specific Stents, Also for Ostial Lesions, and Methods of Application” is directed a balloon or dilatation activated stent particularly for use in a body vessel for specific lesions, particularly in the region of the ostium of a vessel or a bifurcation featuring at least two different stent characteristics as needed for stent treatment. The main portion is predominantly plastically deformable and at least one end portion is elastically deformable and opens to a diameter significantly larger than the diameter of the main portion thereby covering the area of a vessel bifurcation or the ostium and the adjacent vessel wall by conforming to it. The second stent is protruding axially from at least one end (proximal and/or distal) of the first stent. At least one protruding end of the stent assembly is comprised of predominantly self-expanding elastically deformable stent material of shape-memory material forming a flaring end of the protruding end of the stent defining a stent section lying essentially in a surface running perpendicular or obliquely to the longitudinal axis of the remainder of the stent assembly.

U.S. Published Patent Application No. 2014/0277377 to Ischinger and entitled “Oblique Stent” is directed to a stent having a main body with a proximal end and a distal end section having proximal and distal openings used for treatment of lesions in blood vessels and hollow organs, particularly at the ostium of side branches. The stent adapts to the anatomical configuration of a vessel branch by having at least one oblique end section in at least its expanded state.

U.S. Published Patent Application No. 2007/0150042 to Balaji and entitled “Stents with beveled ends and methods of use thereof” is directed to a stent that includes a substantially cylindrical expandable structure having two ends, a locus of points at one of the two ends defining a surface, the surface being beveled with respect to a central axis of the substantially cylindrical expandable structure.

The various references (e.g., referring to Ischinger FIG. 2A, Bourang FIG. 3E, and Balaji FIG. 6) may teach the use of beveled or oblique stents, and/or some types of markers, for treating bifurcations. However, there may be difficulties with ensuring alignment and accurate orientation of beveled stents. Accordingly, there is a need to improve the treatment of a bifurcated vessel in a patient.

This background information is provided to reveal information believed by the applicant to be of possible relevance to the present invention. No admission is necessarily intended, nor should be construed, that any of the preceding information constitutes prior art against the present invention.

### SUMMARY OF THE INVENTION

With the above in mind, embodiments of the present invention are related to improvements in stenting of bifurcated vessels for the treatment of cardiovascular diseases.

Embodiments of the present invention are directed to a system for treating a bifurcated vessel in a patient, the bifurcated vessel including a main vessel and a branch vessel defining an ostium therebetween. The system includes a catheter including an elongated shaft extending from a proximal end to a distal end, and a primary expandable stent delivery balloon, having a proximal portion and a distal portion, and attached to the elongated shaft of the catheter adjacent the distal end thereof. A radially expandable beveled stent, having a longer side and a shorter side, is mounted on the primary expandable stent delivery balloon, the beveled stent is radiopaque and has a distal end and a beveled proximal end with a bevel angle at a leading edge and defined by the longer side and the shorter side of the beveled stent. The catheter includes a guidewire lumen that extends from the proximal end thereof and through the primary expandable stent delivery balloon and to the distal end thereof. The catheter includes an inflation lumen that extends from the proximal end thereof to the primary expandable stent delivery balloon and is in fluid communication therewith to inflate and deflate the primary expandable stent delivery balloon. The primary expandable stent delivery balloon extends between the distal end and the longer side of the beveled proximal end of the beveled stent, the primary expandable stent delivery balloon extends beyond the shorter side of the beveled stent and is configured to transverse the ostium and protrude into the main vessel from the branch vessel during placement of the beveled stent. The primary expandable stent delivery balloon includes three-dimensional (3-D) orientation markers including a triangular shaped proximal radiopaque marker that is positioned on the proximal portion of the primary expandable stent delivery balloon and configured to demarcate a triangle defined by a difference between the longer side and the shorter side at the leading edge at the beveled proximal end of the beveled stent mounted on the primary expandable stent delivery balloon, and a distal radiopaque marker that is positioned at the distal portion of the primary expandable stent delivery balloon and configured to identify, at the distal end of the beveled stent, the longer side of the beveled stent.

Additionally, and/or alternatively, in certain embodiments a hypotenuse of the triangular shaped proximal radiopaque marker demarcates the ostium of the branch vessel.

Additionally, and/or alternatively, in certain embodiments the distal radiopaque marker is an arrow-shaped distal radiopaque marker.

Additionally, and/or alternatively, in certain embodiments the bevel angle is less than 90 degrees to conform to the ostium of the branch vessel arising with less than a 90-degree angle from the main vessel.

Additionally, and/or alternatively, in certain embodiments a secondary expandable balloon, having a proximal portion and a distal portion, is attached to an upper side of the elongated shaft of the catheter that corresponds to the longer side of the beveled stent, and proximally adjacent to the proximal portion of the primary expandable stent delivery balloon that includes the triangular shaped proximal radiopaque marker positioned thereon. Here, the catheter includes a secondary inflation lumen that extends from the proximal end thereof to the secondary expandable balloon and is in fluid communication therewith to inflate and deflate the secondary expandable balloon independently from the primary expandable stent delivery balloon.

Additionally, and/or alternatively, in certain embodiments the secondary expandable balloon is inflated with contrast and configured to remain outside the ostium of the branch vessel to aid in placement of the beveled stent.

Additionally, and/or alternatively, in certain embodiments the elongated shaft of the catheter includes an angulation just proximal to the beveled stent mounted on the primary expandable stent delivery balloon, and the angulation corresponds to an angle between the main vessel and the branch vessel.

Additionally, and/or alternatively, in certain embodiments the radially expandable beveled stent is configured to be radially expandable, via inflation of the primary expandable stent delivery balloon, from a non-deployed state to a deployed state. A difference in length (ΔL) between the longer side and the shorter side of the radially expandable beveled stent is calculated based upon the angle (Θ), defined as 90 minus a determined angle of the branch vessel, and the radius (r) of the branch vessel using an equation:

ΔL=(tangent Θ)×r.

Another example embodiment of the present invention is directed to system for treating a bifurcated vessel in a patient, the bifurcated vessel including a main vessel and a branch vessel defining an ostium therebetween arising with less than a 90-degree angle from the main vessel. The system includes a catheter including an elongated shaft extending from a proximal end to a distal end, and a primary expandable stent delivery balloon attached to the elongated shaft of the catheter adjacent the distal end thereof. A radially expandable beveled stent, having a longer side and a shorter side, is mounted on the primary expandable stent delivery balloon, the beveled stent is radiopaque and has a distal end and a beveled proximal end with a bevel angle at a leading edge which is configured to conform to the ostium of the branch vessel arising with less than the 90-degree angle from the main vessel. The catheter includes an inflation lumen that extends from the proximal end thereof to the primary expandable stent delivery balloon and is in fluid communication therewith to inflate and deflate the primary expandable stent delivery balloon. The primary expandable stent delivery balloon extends between the distal end and the longer side of the beveled proximal end of the beveled stent. The primary expandable stent delivery balloon includes three-dimensional (3-D) orientation markers including a triangular shaped proximal radiopaque marker that is positioned on the primary expandable stent delivery balloon and configured to demarcate a triangle defined by a difference between the longer side and the shorter side at the leading edge at the beveled proximal end of the beveled stent mounted on the primary expandable stent delivery balloon. A distal radiopaque marker is positioned on the primary expandable stent delivery balloon and configured to identify, at the distal end of the beveled stent, the longer side of the beveled stent.

Additionally, and/or alternatively, in certain embodiments the primary expandable stent delivery balloon extends beyond the shorter side of the beveled stent and is configured to transverse the ostium and protrude into the main vessel from the branch vessel during placement of the beveled stent.

Another example embodiment of the present invention is directed to a method for treating a bifurcated vessel in a patient, the bifurcated vessel including a main vessel and a branch vessel defining an ostium therebetween. The method incudes: providing a catheter including an elongated shaft extending from a proximal end to a distal end; attaching a primary expandable stent delivery balloon, having a proximal portion and a distal portion, to the elongated shaft of the catheter adjacent the distal end thereof; and mounting a radially expandable beveled stent, having a longer side and a shorter side, on the primary expandable stent delivery balloon, the beveled stent is radiopaque and has a distal end and a beveled proximal end with a bevel angle at a leading edge and defined by the longer side and the shorter side of the beveled stent. The method includes: guiding the catheter using a guide wire within a guidewire lumen that extends from the proximal end of the catheter through the primary expandable stent delivery balloon and to the distal end thereof; and inflating and deflating the primary expandable stent delivery balloon via an inflation lumen that extends from the proximal end of the catheter to the primary expandable stent delivery balloon and is in fluid communication therewith. The primary expandable stent delivery balloon extends between the distal end and the longer side of the beveled proximal end of the beveled stent, the primary expandable stent delivery balloon extends beyond the shorter side of the beveled stent to transverse the ostium and protrude into the main vessel from the branch vessel during placement of the beveled stent. The method further includes orienting the primary expandable stent delivery balloon using three-dimensional (3-D) orientation markers that include a triangular shaped proximal radiopaque marker positioned on the proximal portion of the primary expandable stent delivery balloon to demarcate a triangle defined by a difference between the longer side and the shorter side at the leading edge at the beveled proximal end of the beveled stent mounted on the primary expandable stent delivery balloon, and a distal radiopaque marker positioned at the distal portion of the primary expandable stent delivery balloon to identify, at the distal end of the beveled stent, the longer side of the beveled stent.

These and other embodiments enhance the alignment and accurate orientation of beveled stents during the treatment of bifurcated vessels for the treatment of cardiovascular diseases.

### BRIEF DESCRIPTION OF THE DRAWINGS

Some embodiments of the present invention are illustrated as an example and are not limited by the figures of the accompanying drawings, in which like references may indicate similar elements,

FIG. 1 is a longitudinal cross-sectional view of a bifurcated main vessel with a conventional non-beveled stent placed at a 90-degree branch vessel according to the prior art.

FIGS. 2A and 2B are longitudinal cross-sectional views of a bifurcated main vessel with a conventional non-beveled stent placed at a less-than-90-degree branch vessel according to the prior art. In FIG. 2A the stent placement misses a triangular portion of the branch vessel at the ostium. In FIG. 2B the stent placement protrudes into the main vessel.

FIG. 3 is a side view illustration of the stent delivery balloon catheter including a beveled stent positioned on a stent delivery balloon that includes 3-D orientation markers in accordance with embodiments of the invention.

FIG. 4 is a cross-sectional view of a stent delivery balloon catheter of FIG. 3 and including a guidewire lumen and an inflation lumen in accordance with embodiments of the present invention.

FIGS. 5A and 5B are longitudinal cross-sectional views of a bifurcated main vessel with the beveled stent of FIG. 3 being maneuvered to be deployed at a less-than-90-degree branch vessel. In FIG. 5A the beveled stent is being steered toward the branch vessel via the ostium. In FIG. 5B the beveled stent is being oriented using the 3-D orientation markers to align with the less-than-90-degree angle at the ostium.

FIG. 5C is a diagram illustrating the calculation of the difference in length (ΔL) between the longer side and the shorter side of the radially expandable beveled stent of FIGS. 5A and 5B based upon the angle (Θ) and the radius (r) of the branch vessel, in accordance with embodiments of the present invention.

FIG. 6 is a longitudinal cross-sectional view of the bifurcated main vessel with the beveled stent of FIGS. 5A and 5B deployed and aligned with the less-than-90-degree angle at the ostium in accordance with embodiments of the present invention.

FIG. 7 is a longitudinal cross-sectional view of the bifurcated main vessel with another embodiment of the stent delivery balloon catheter including the beveled stent of FIG. 3 positioned on the balloon and further including a secondary expandable balloon and an angulation in the elongated shaft of the catheter in accordance with features of the present invention.

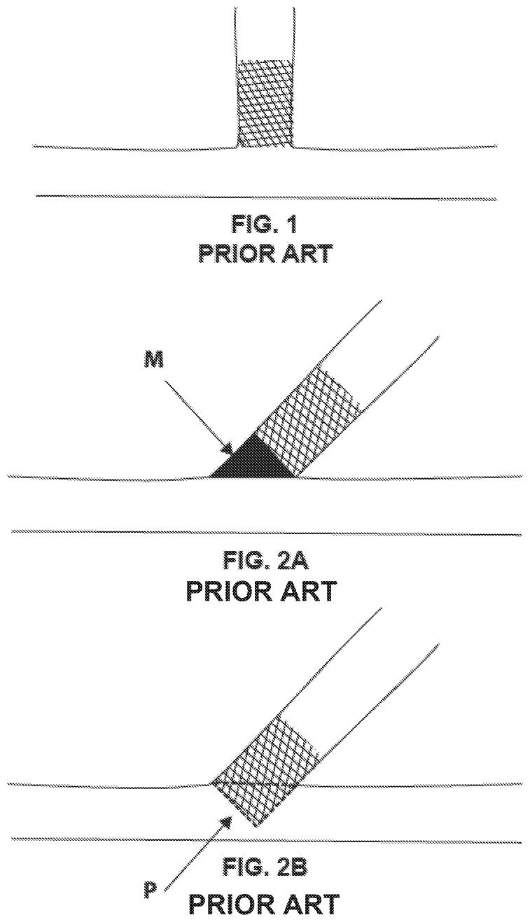

### DETAILED DESCRIPTION OF THE INVENTION

The present invention will now be described more fully hereinafter with reference to the accompanying drawings, in which preferred embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art. Those of ordinary skill in the art realize that the following descriptions of the embodiments of the present invention are illustrative and are not intended to be limiting in any way. Other embodiments of the present invention will readily suggest themselves to such skilled persons having the benefit of this disclosure. Like numbers refer to like elements throughout.

Although the following detailed description contains many specifics for the purposes of illustration, anyone of ordinary skill in the art will appreciate that many variations and alterations to the following details are within the scope of the invention. Accordingly, the following embodiments of the invention are set forth without any loss of generality to, and without imposing limitations upon, the claimed invention.

In this detailed description of the present invention, a person skilled in the art should note that directional terms, such as “above,” “below,” “upper,” “lower,” and other like terms are used for the convenience of the reader in reference to the drawings. Also, a person skilled in the art should notice this description may contain other terminology to convey position, orientation, and direction without departing from the principles of the present invention.

Furthermore, in this detailed description, a person skilled in the art should note that quantitative qualifying terms such as “generally,” “substantially,” “mostly,” and other terms are used, in general, to mean that the referred to object, characteristic, or quality constitutes a majority of the subject of the reference. The meaning of any of these terms is dependent upon the context within which it is used, and the meaning may be expressly modified.

The present invention will now be described more fully hereinafter with reference to the accompanying drawings, in which preferred embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art. Those of ordinary skill in the art realize that the following descriptions of the embodiments of the present invention are illustrative and are not intended to be limiting in any way. Other embodiments of the present invention will readily suggest themselves to such skilled persons having the benefit of this disclosure. Like numbers refer to like elements throughout.

Before describing the present disclosure in detail, it is to be understood that this disclosure is not limited to parameters of the particularly exemplified systems, methods, apparatus, products, processes, and/or kits, which may, of course, vary. It is also to be understood that the terminology used herein is only for the purpose of describing particular embodiments of the present disclosure, and is not necessarily intended to limit the scope of the disclosure in any particular manner. Thus, while the present disclosure will be described in detail with reference to specific embodiments, features, aspects, configurations, etc., the descriptions are illustrative and are not to be construed as limiting the scope of the claimed invention. Various modifications can be made to the illustrated embodiments, features, aspects, configurations, etc. without departing from the spirit and scope of the invention as defined by the claims. Thus, while various aspects and embodiments have been disclosed herein, other aspects and embodiments are contemplated.

Unless defined otherwise, all technical and scientific terms used herein have the same meaning as commonly understood by one of ordinary skill in the art to which the present disclosure pertains. While a number of methods and materials similar or equivalent to those described herein can be used in the practice of the present disclosure, only certain exemplary materials and methods are described herein.

Various aspects of the present disclosure, including devices, systems, methods, etc., may be illustrated with reference to one or more exemplary embodiments or implementations. As used herein, the terms “embodiment,” “alternative embodiment” and/or “exemplary implementation” means “serving as an example, instance, or illustration,” and should not necessarily be construed as preferred or advantageous over other embodiments or implementations disclosed herein. In addition, reference to an “implementation” of the present disclosure or invention includes a specific reference to one or more embodiments thereof, and vice versa, and is intended to provide illustrative examples without limiting the scope of the invention, which is indicated by the appended claims rather than by the following description.

It will be noted that, as used in this specification and the appended claims, the singular forms “a,” “an” and “the” include plural referents unless the content clearly dictates otherwise. Thus, for example, reference to a “sensor” includes one, two, or more sensors.

As used throughout this application the words “can” and “may” are used in a permissive sense (i.e., meaning having the potential to), rather than the mandatory sense (i.e., meaning must). Additionally, the terms “including,” “having,” “involving,” “containing,” “characterized by,” variants thereof (e.g., “includes,” “has,” and “involves,” “contains,” etc.), and similar terms as used herein, including the claims, shall be inclusive and/or open-ended, shall have the same meaning as the word “comprising” and variants thereof (e.g., “comprise” and “comprises”), and do not exclude additional, un-recited elements or method steps, illustratively.

Various aspects of the present disclosure can be illustrated by describing components that are coupled, attached, connected, and/or joined together. As used herein, the terms “coupled”, “attached”, “connected,” and/or “joined” are used to indicate either a direct connection between two components or, where appropriate, an indirect connection to one another through intervening or intermediate components. In contrast, when a component is referred to as being “directly coupled”, “directly attached”, “directly connected,” and/or “directly joined” to another component, no intervening elements are present or contemplated. Thus, as used herein, the terms “connection,” “connected,” and the like do not necessarily imply direct contact between the two or more elements. In addition, components that are coupled, attached, connected, and/or joined together are not necessarily (reversibly or permanently) secured to one another. For instance, coupling, attaching, connecting, and/or joining can comprise placing, positioning, and/or disposing the components together or otherwise adjacent in some implementations.

As used herein, directional and/or arbitrary terms, such as “top,” “bottom,” “front,” “back,” “left,” “right,” “up,” “down,” “upper,” “lower,” “inner,” “outer,” “internal,” “external,” “interior,” “exterior,” “proximal,” “distal” and the like can be used solely to indicate relative directions and/or orientations and may not otherwise be intended to limit the scope of the disclosure, including the specification, invention, and/or claims.

Where possible, like numbering of elements have been used in various figures. In addition, similar elements and/or elements having similar functions may be designated by similar numbering (e.g., element “10” and element “210.”) Furthermore, alternative configurations of a particular element may each include separate letters appended to the element number. Accordingly, an appended letter can be used to designate an alternative design, structure, function, implementation, and/or embodiment of an element or feature without an appended letter. Similarly, multiple instances of an element and or sub-elements of a parent element may each include separate letters appended to the element number. In each case, the element label may be used without an appended letter to generally refer to instances of the element or any one of the alternative elements. Element labels including an appended letter can be used to refer to a specific instance of the element or to distinguish or draw attention to multiple uses of the element. However, element labels including an appended letter are not meant to be limited to the specific and/or particular embodiment(s) in which they are illustrated. In other words, reference to a specific feature in relation to one embodiment should not be construed as being limited to applications only within said embodiment.

It will also be appreciated that where a range of values (e.g., less than, greater than, at least, and/or up to a certain value, and/or between two recited values) is disclosed or recited, any specific value or range of values falling within the disclosed range of values is likewise disclosed and contemplated herein.

It is also noted that systems, methods, apparatus, devices, products, processes, compositions, and/or kits, etc., according to certain embodiments of the present invention may include, incorporate, or otherwise comprise properties, features, aspects, steps, components, members, and/or elements described in other embodiments disclosed and/or described herein. Thus, reference to a specific feature, aspect, steps, component, member, element, etc. in relation to one embodiment should not be construed as being limited to applications only within said embodiment. In addition, reference to a specific benefit, advantage, problem, solution, method of use, etc. in relation to one embodiment should not be construed as being limited to applications only within said embodiment.

The headings used herein are for organizational purposes only and are not meant to be used to limit the scope of the description or the claims. To facilitate understanding, like reference numerals have been used, where possible, to designate like elements common to the figures.

Stents are generally tubular devices for insertion into body lumens. Balloon expandable stents require mounting over a stent delivery balloon, positioning, and then inflation of the balloon to expand the stent radially outward. Self-expanding stents expand into place when unconstrained, without requiring assistance from a balloon. A self-expanding stent is biased so as to expand upon release from the delivery catheter. Some stents may be characterized as hybrid stents which have some characteristics of both self-expandable and balloon expandable stents.

Stents may be constructed from a variety of materials including metals such as stainless steel and nickel-titanium alloy, or shape memory polymers, etc. Stents may also be formed in a variety of manners, for example, by etching or cutting the stent pattern from a tube or section of stent material. A sheet of stent material may be cut or etched according to a desired stent pattern whereupon the sheet may be rolled or otherwise formed into the desired tubular or bifurcated tubular shape of the stent. Or, one or more wires or ribbons of stent material may be braided or otherwise formed into a desired shape and pattern. Stents coated with drugs that interrupt re-narrowing are called drug-eluting stents.

The stent may have a collapsed configuration suitable for delivery to the bifurcation, and a radially expanded configuration adapted to engage and support a vessel wall at the bifurcation or adjacent the bifurcation. Each of the stents may be crimped to its respective expandable member to prevent ejection of the stent during advancement through a patient's vasculature.

A vessel having a stenosis may be viewed as an inwardly protruding arcuate addition of hardened material to a cylindrical vessel wall, where the stenosed region presents a somewhat rigid body attached along, and to, the elastic wall. The stenosis presents resistance to any expansion of the vessel in the region bridged by the stenosis. Stenoses vary in composition, for example, in the degree of calcification, and therefore vary in properties as well.

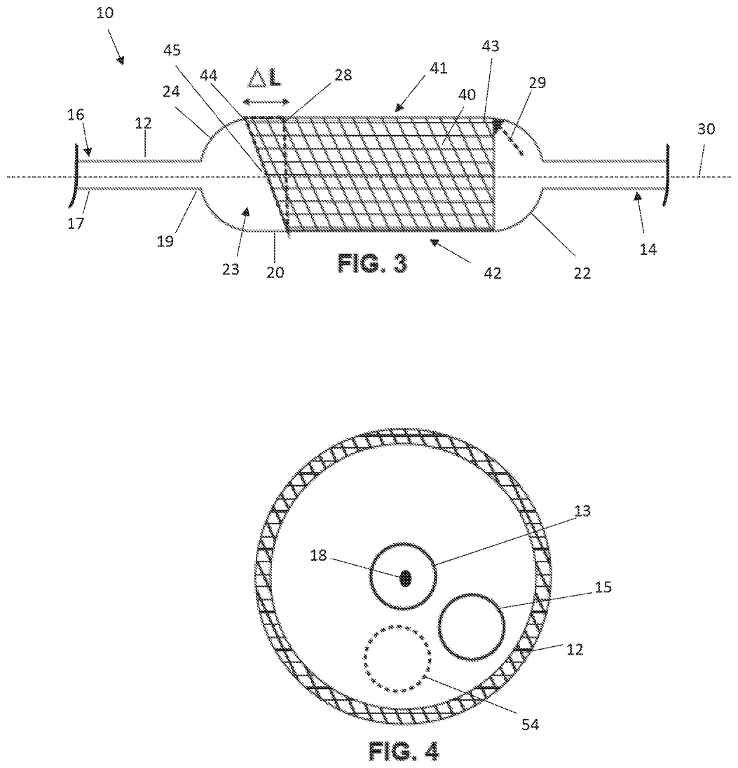

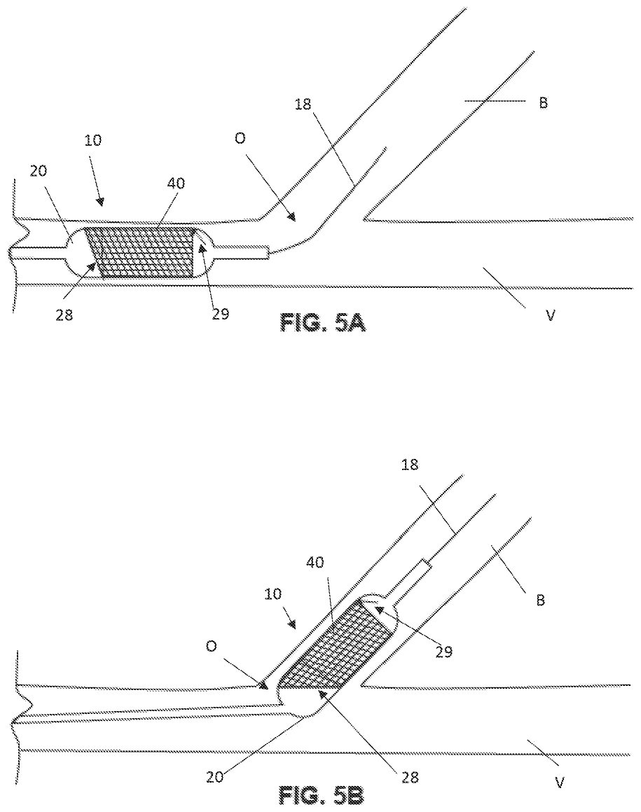

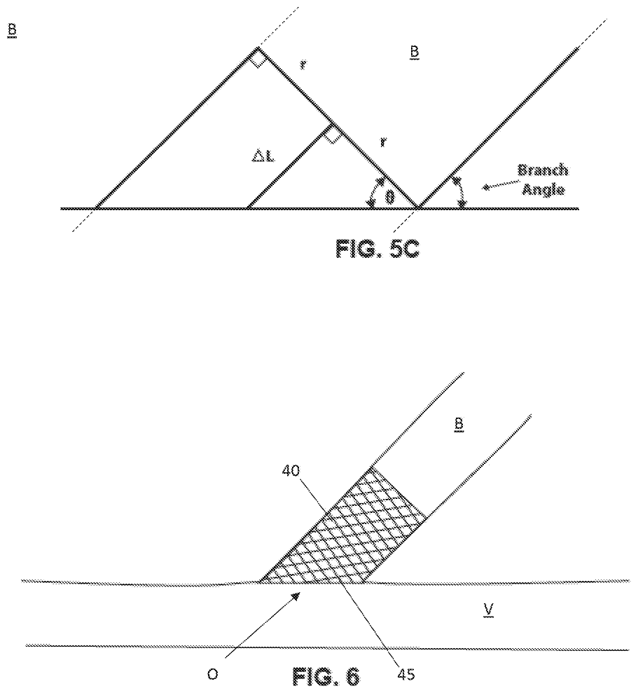

With initial reference to FIGS. 3, 4, 5A and 5B, an embodiment of the invention, a system for treating a bifurcated vessel in a patient, will be described. As described above, the bifurcated vessel includes a main vessel V and a branch vessel B defining an ostium O therebetween. The branch vessel B arises at an angle less than 90 degrees from the main vessel V. A stent delivery balloon catheter 10 including a beveled stent 40 positioned on a stent delivery balloon 20 that includes 3-D orientation markers 28 and 29. FIG. 3 is a side view illustration of the stent delivery balloon catheter including the beveled stent positioned on the stent delivery balloon that includes 3-D orientation markers. FIG. 4 is a cross-sectional view of the stent delivery balloon catheter and includes a guidewire lumen and an inflation lumen. FIGS. 5A and 5B are longitudinal cross-sectional views of a bifurcated main vessel with the beveled stent being maneuvered to be deployed at a less-than-90-degree branch vessel. In FIG. 5A the beveled stent is being steered toward the branch vessel via the ostium. In FIG. 5B the beveled stent is being oriented using the 3-D orientation markers to align with the less-than-90-degree angle at the ostium.

Catheter 10 includes a shaft 12 having a shaft distal end 14 and a shaft proximal end 16 along a longitudinal axis 30. In some embodiments, shaft 12 is a guidewire shaft having a guidewire lumen 13 suitable for holding a guidewire 18 therein. Guidewire lumen 13 may extend along a length of shaft 12 from shaft distal end 14 to shaft proximal end 16 for an “over the wire” configuration. Alternatively, a guidewire exit point may be positioned at a point along shaft 12 for a “rapid exchange” configuration (not illustrated here but well known to those skilled in the art). Alternatively, shaft 12 may not include a guidewire lumen and instead, a fixed wire may be attached to shaft 12 at shaft distal end 14. The guidewire 18 is made of a relatively stiffer proximal part and a shapeable less stiff distal part that makes it steerable, as would be appreciated by those skilled in the art.

An expandable stent delivery balloon 20 (also referred to as a primary expandable stent delivery balloon) is positioned on shaft 12. Typically, balloon 20 is positioned at or near shaft distal end 14. Shaft 12 may include a stiff segment 17, extending from a point proximal to balloon 20 to the shaft proximal end 16, and a flexible segment 19 having the balloon 20 positioned thereon. Stiff segment 17 may include a relatively stiff wire or any other material which has enough stiffness to transmit torque forces from one end to the other. Flexible segment 19 includes a flexible material that can bend sufficiently to maneuver along twist and turns within the vessel. Balloon 20 is positioned on flexible segment 19. Balloon 20 may be an inflatable balloon, or may be expandable by other methods, e.g. removal of a sheath. The balloon 20 may be made of a compliant, semi-compliant or noncompliant material, and with a low-profile.

The balloon 20 is typically an elongate tube-shaped structure sized to traverse the vessel bifurcation (i.e., extend from proximal of, and to distal of, the ostium O of branch vessel). Balloon 20 may have a distal portion 22 and a proximal portion 24 along longitudinal axis 30. Balloon 20 encloses a space 23 that may be filled with an inflation fluid (e.g. contrast), as is known to those skilled in the art, to inflate balloon 20. The catheter 10 may include an inflation lumen 15 that is in fluid communication with the balloon 20 for inflating and deflating the balloon 20.

The radially expandable beveled stent 40, has a longer side 41 and a shorter side 42, and is mounted on the primary expandable stent delivery balloon 20. The beveled stent 40 is radiopaque and has a distal end 43 and a beveled proximal end 44 with a bevel angle at a leading edge 45 and defined by the longer side 41 and the shorter side 42 of the beveled stent 40. The bevel angle is less than 90 degrees to conform to the ostium O of the branch vessel B arising with less than a 90-degree angle from the main vessel V.

The guidewire lumen 13 extends from the proximal end 16 of the catheter 10 through the primary expandable stent delivery balloon 20 and to the distal end 14 thereof. The inflation lumen 15 that extends from the proximal end 16 to the primary expandable stent delivery balloon 20.

The stent 40 mounted on the balloon 20 may be made of a metal that is stainless steel or an alloy such as cobalt chromium or titanium or any other alloys. Platinum may be added to improve radiopacity. The stent 40 may be made of slotted tube, wire design or modules. The stent material and design may be made to allow for thin struts without compromising radial strength and with optimal radiopacity and other properties to optimize pushability, deliverability and minimize vessel injury. The stent 40 may or may not be covered by polymers, drugs, etc. to minimize vessel injury and neointimal proliferation and to be optimal for vessel healing. These materials including the stent may be partially or completely bio-absorbable. The stent 40 and the balloon 20 may be made in different lengths and predetermined deployment diameters. Delta L may also come in different pre-specified lengths. Those skilled in the art will appreciate, however, that these may vary based on the vessel size, angle, and lesion length.

The primary expandable stent delivery balloon 20 extends between the distal end 43 and the longer side 41 of the beveled proximal end 44 of the beveled stent 40. The primary expandable stent delivery balloon 20 extends beyond the shorter side 42 of the beveled stent 20 and is configured to transverse the ostium O and protrude into the main vessel V from the branch vessel B during placement of the beveled stent 40.

The primary expandable stent delivery balloon 20 includes three-dimensional (3-D) orientation markers 28, 29. A triangular shaped proximal radiopaque marker 28 is positioned on the proximal portion 24 of the primary expandable stent delivery balloon 20 and is configured to demarcate a triangle defined by a difference ΔL between the longer side 41 and the shorter side 42 at the leading edge 45 at the beveled proximal end 44 of the beveled stent 40 mounted on the primary expandable stent delivery balloon 20. A distal radiopaque marker 29, such as an arrow, is positioned at the distal portion 22 of the primary expandable stent delivery balloon 20 and configured to identify, at the distal end 43 of the beveled stent 40, the longer side 41 of the beveled stent 40.

The 3-D orientation markers 28, 29 are easily seen by imaging during the procedure to place the stent 40 at the branch vessel B of the patient. The 3-D orientation markers 28, 29 ensure accurate three-dimensional orientation using two-dimensional imaging such as an angiogram. The 3-D orientation markers 28, 29 are superimposed on the stent 40 during imaging. The triangular shaped proximal radiopaque marker 28 is superimposed on proximal end 44 of the stent 40, and distal marker 29, illustrated as an arrow with the arrowhead pointing to the long side 41 of the stent 40, is superimposed on the distal end 43 of the stent 40. This will aid with 3-D orientation of the stent 40 to make sure that the ostium O of the branch vessel B is precisely covered by the stent 40 with no part of the stent 40 protruding into the main vessel V. The 3-D orientation markers 28, 29 may be made of radiopaque material such as platinum, and mark the two ends 43, 44 of the stent 40 accurately. As best illustrated in FIG. 5B, the hypotenuse of the triangular shaped proximal radiopaque marker 28 should be lined up with the true ostium O of the branch vessel B. So, the hypotenuse of the triangular marker 28 demarcates the ostium O of the branch vessel B.

With additional reference to FIG. 5C, a trigonometric method to calculate the length ΔL of the long side 41 at the leading edge 45 of the non-deployed stent 40 using angle theta (θ) and the radius (r) of the branch vessel B will be described. The method is based on the formula of a right triangle: tangent θ=opposite side/adjacent side, Angle θ is calculated as 90° minus the branch angle, and the adjacent side of the triangle will be the radius r of the branch vessel B (which can be estimated visually or via intravascular ultrasound, for example). With these two known values, the opposite side (which is the leading edge 45 of the stent 40) can be calculated. The radius will be used rather than the diameter assuming that the non-deployed stent 40 is in the approximate middle of the vessel branch B as shown in FIG. 5C.

Accordingly, as described, the radially expandable beveled stent 40 is configured to be radially expandable, via inflation of the primary expandable stent delivery balloon 20, from the non-deployed state (FIG. 5B) to a deployed state (FIG. 6). So, the difference in length ΔL between the longer side 41 and the shorter side 42 of the radially expandable beveled stent 40 is calculated based upon the angle θ, defined as 90 minus a determined angle of the branch vessel, and the radius r of the branch vessel B using the equation:

ΔL=(tangent θ)×r.

Referring to FIG, 7, another embodiment of the system for treating a bifurcated vessel in a patient, will be described. As described above, the bifurcated vessel includes a main vessel V and a branch vessel B defining an ostium O therebetween. The branch vessel B arises at an angle less than 90 degrees from the main vessel V. A stent delivery balloon catheter 50 includes the beveled stent 40 positioned on the stent delivery balloon 20 and includes the 3-D orientation markers 28 and 29.

A secondary expandable balloon 52, having a proximal portion and a distal portion, is attached to an upper side of the elongated shaft 12 of the catheter 50 that corresponds to the longer side 41 of the beveled stent. The secondary expandable balloon 52 is proximally adjacent to the proximal portion or end 44 of the primary expandable stent delivery balloon 20 that includes the triangular shaped proximal radiopaque marker 28 positioned thereon. In this embodiment, the shaft 12 of the catheter 50 includes a secondary inflation lumen 54 (FIG. 4) that extends from the proximal end thereof to the secondary expandable balloon 52 and is in fluid communication therewith to inflate and deflate the secondary expandable balloon 52 independently from the primary expandable stent delivery balloon 40.

As such, the secondary expandable balloon 52 that will help precise placement of the stent 40. The secondary expandable balloon 52 will be filled with contrast but is relatively smaller than the primary expandable stent delivery balloon 20, but big enough to be visualized during imaging. For example, if the secondary expandable balloon 52 appears to be going inside the branch vessel B then the stent 40 is too far in (i.e., not aligned with the true branch ostium O), and if the secondary expandable balloon 52 is too far proximal to the branch vessel B then the stent 40 will be too proximal to the ostium O and protruding in the main vessel V.

The secondary expandable balloon 52 of this embodiment can also be used for accurate stent placement in general outside the specific situation of branch stenting, and can also be used for stenting of branches arising at 90° angle, for example.

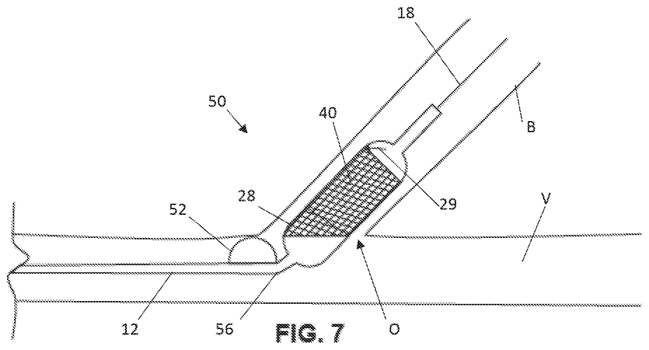

Also referring to FIG. 7, the elongated shaft 12 of the catheter 10 may include an angulation 56 just proximal to the beveled stent 40 mounted on the primary expandable stent delivery balloon 20. The angulation 52 may correspond to the branch angle between the main vessel V and the branch vessel B. This feature will also help with precise and accurate placement of the stent 40. The angulation 56 of the shaft 12 of the catheter 50 just proximal to the stent 40 mounted on the primary expandable stent delivery balloon 20 may be substantially equal to the branch angle. As such, the system may be provided with different catheters having pre-set angles such as 80°, 70°, 60° etc. This angulation 56 will help avoid any potential torque or pull on the stent 40 before being deployed and will allow more “relaxed” positioning of the stent 40 for more accurate placement. Again, the guidewire 18 is made of a relatively stiffer proximal part and a shapeable less stiff distal part that makes it steerable, as would be appreciated by those skilled in the art. Thus, delivering or guiding the catheter 50 with angulation 56 should not be an issue as it may be straightened by the proximal (more stiff) part of the guidewire 18 and then more relaxed at the distal (less stiff) part of the guidewire 18 allowing it to take its shape.

Some of the illustrative aspects of the present invention may be advantageous in solving the problems herein described and other problems not discussed which are discoverable by a skilled artisan.

While the above description contains much specificity, these should not be construed as limitations on the scope of any embodiment, but as exemplifications of the presented embodiments thereof. Many other ramifications and variations are possible within the teachings of the various embodiments. While the invention has been described with reference to exemplary embodiments, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best or only mode contemplated for carrying out this invention, but that the invention will include all embodiments falling within the scope of the appended claims. Also, in the drawings and the description, there have been disclosed exemplary embodiments of the invention and, although specific terms may have been employed, they are unless otherwise stated used in a generic and descriptive sense only and not for purposes of limitation, the scope of the invention therefore not being so limited. Moreover, the use of the terms first, second, etc. do not denote any order or importance, but rather the terms first, second, etc. are used to distinguish one element from another, Furthermore, the use of the terms a, an, etc. do not denote a limitation of quantity, but rather denote the presence of at least one of the referenced items.

Thus, the scope of the invention should be determined by the appended claims and their legal equivalents, and not by the examples given.

What is claimed is:

1. A system for treating a bifurcated vessel in a patient, the bifurcated vessel including a main vessel and a branch vessel defining an ostium therebetween, the system comprising:
a catheter including an elongated shaft extending from a proximal end to a distal end;
a primary expandable stent delivery balloon, having a proximal portion and a distal portion, and attached to the elongated shaft of the catheter adjacent the distal end thereof;
a radially expandable beveled stent having a longer side and a shorter side, and being mounted on the primary expandable stent delivery balloon, the beveled stent being radiopaque and having a distal end and a beveled proximal end with a bevel angle at a leading edge and defined by the longer side and the shorter side of the beveled stent;
wherein the catheter includes a guidewire lumen that extends from the proximal end through the primary expandable stent delivery balloon and to the distal end thereof;
wherein the catheter includes an inflation lumen that extends from the proximal end to the primary expandable stent delivery balloon and is in fluid communication therewith to inflate and deflate the primary expandable stent delivery balloon;
wherein the primary expandable stent delivery balloon extends between the distal end and the longer side of the beveled proximal end of the beveled stent;
wherein the primary expandable stent delivery balloon extends beyond the shorter side of the beveled stent and is configured to transverse the ostium and protrude into the main vessel from the branch vessel during placement of the beveled stent; and
wherein the primary expandable stent delivery balloon includes three-dimensional (3-D) orientation markers including:
a triangular shaped proximal radiopaque marker that is positioned on the proximal portion of the primary expandable stent delivery balloon and configured to demarcate a triangle defined by a difference between the longer side and the shorter side at the leading edge at the beveled proximal end of the beveled stent mounted on the primary expandable stent delivery balloon, and
a distal radiopaque marker that is positioned at the distal portion of the primary expandable stent delivery balloon and configured to identify, at the distal end of the beveled stent, the longer side of the beveled stent.

2. The system according to claim 1 wherein a hypotenuse of the triangular shaped proximal radiopaque marker demarcates the ostium of the branch vessel.

3. The system according to claim 1 wherein the distal radiopaque marker comprises an arrow-shaped distal radiopaque marker.

4. The system according to claim 1 wherein the bevel angle is less than 90 degrees to conform to the ostium of the branch vessel arising with less than a 90-degree angle from the main vessel.

5. The system according to claim 1 further comprising:
a secondary expandable balloon, having a proximal portion and a distal portion, and attached to an upper side of the elongated shaft of the catheter that corresponds to the longer side of the beveled stent, and proximally adjacent to the proximal portion of the primary expandable stent delivery balloon that includes the triangular shaped proximal radiopaque marker positioned thereon; and
the catheter includes a secondary inflation lumen that extends from the proximal end to the secondary expandable balloon and is in fluid communication therewith to inflate and deflate the secondary expandable balloon independently from the primary expandable stent delivery balloon.

6. The system according to claim 5 wherein the secondary expandable balloon is filled with contrast and configured to remain outside the ostium of the branch vessel to aid in placement of the beveled stent.

7. The system according to claim 1 wherein the elongated shaft of the catheter includes an angulation proximal to the beveled stent mounted on the primary expandable stent delivery balloon; and wherein the angulation corresponds to an angle between the main vessel and the branch vessel.

8. The system according to claim 1 wherein the radially expandable beveled stent is configured to be radially expandable, via inflation of the primary expandable stent delivery balloon, from a non-deployed state to a deployed state; wherein a difference in length (ΔL) between the longer side and the shorter side of the radially expandable stent is calculated based upon the angle (Θ), defined as 90 minus a determined angle of the branch vessel, and the radius (r) of the branch vessel using an equation:
ΔL=(tangent Θ)×r.

9. A system for treating a bifurcated vessel in a patient, the bifurcated vessel including a main vessel and a branch vessel defining an ostium therebetween arising with less than a 90-degree angle from the main vessel, the system comprising:
a catheter including an elongated shaft extending from a proximal end to a distal end;
a primary expandable stent delivery balloon attached to the elongated shaft of the catheter adjacent the distal end thereof;
a radially expandable beveled stent, having a longer side and a shorter side, and mounted on the primary expandable stent delivery balloon, the beveled stent is radiopaque and has a distal end and a beveled proximal end with a bevel angle at a leading edge which is configured to conform to the ostium of the branch vessel arising with less than the 90-degree angle from the main vessel;
wherein the catheter includes an inflation lumen that extends from the proximal end to the primary expandable stent delivery balloon and is in fluid communication therewith to inflate and deflate the primary expandable stent delivery balloon;
wherein the primary expandable stent delivery balloon extends between the distal end and the longer side of the beveled proximal end of the beveled stent; and
wherein the primary expandable stent delivery balloon includes three-dimensional (3-D) orientation markers including:
a triangular shaped proximal radiopaque marker that is positioned on the primary expandable stent delivery balloon and configured to demarcate a triangle defined by a difference between the longer side and the shorter side at the leading edge at the beveled proximal end of the beveled stent mounted on the primary expandable stent delivery balloon, and
a distal radiopaque marker that is positioned on the primary expandable stent delivery balloon and configured to identify, at the distal end of the beveled stent, the longer side of the beveled stent.

10. The system according to claim 9 wherein a hypotenuse of the triangular shaped proximal radiopaque marker demarcates the ostium of the branch vessel; and wherein the distal radiopaque marker comprises an arrow-shaped distal radiopaque marker.

11. The system according to claim 9 wherein the primary expandable stent delivery balloon extends beyond the shorter side of the beveled stent and is configured to transverse the ostium and protrude into the main vessel from the branch vessel during placement of the beveled stent.

12. The system according to claim 9 further comprising:
a secondary expandable balloon attached to an upper side of the elongated shaft of the catheter that corresponds to the longer side of the beveled stent, and proximally adjacent to the proximal portion of the primary expandable stent delivery balloon that includes the triangular shaped proximal radiopaque marker positioned thereon;
the catheter includes a secondary inflation lumen that extends from the proximal end to the secondary expandable balloon and is in fluid communication therewith to inflate and deflate the secondary expandable balloon independently from the primary expandable stent delivery balloon; and
the secondary expandable balloon is filled with contrast and configured to remain outside the ostium of the branch vessel to aid in placement of the beveled stent.

13. The system according to claim 9 wherein the elongated shaft of the catheter includes an angulation just proximal to the beveled stent mounted on the primary expandable stent delivery balloon; and wherein the angulation corresponds to an angle between the main vessel and the branch vessel.

14. A method for treating a bifurcated vessel in a patient, the bifurcated vessel including a main vessel and a branch vessel defining an ostium therebetween, the method comprising:
providing a catheter including an elongated shaft extending from a proximal end to a distal end;
attaching a primary expandable stent delivery balloon having a proximal portion and a distal portion to the elongated shaft of the catheter adjacent the distal end thereof;
mounting a radially expandable beveled stent having a longer side and a shorter side on the primary expandable stent delivery balloon;
guiding the catheter using a guide wire within a guidewire lumen that extends from the proximal end of the catheter through the primary expandable stent delivery balloon and to the distal end thereof;
inflating and deflating the primary expandable stent delivery balloon via an inflation lumen that extends from the proximal end of the catheter to the primary expandable stent delivery balloon and is in fluid communication therewith;
wherein the primary expandable stent delivery balloon extends between the distal end and the longer side of the beveled proximal end of the beveled stent;
wherein the primary expandable stent delivery balloon extends beyond the shorter side of the beveled stent to transverse the ostium and protrude into the main vessel from the branch vessel during placement of the beveled stent; and
orienting the primary expandable stent delivery balloon using three-dimensional (3-D) orientation markers that include:
a triangular shaped proximal radiopaque marker positioned on the proximal portion of the primary expandable stent delivery balloon to demarcate a triangle defined by a difference between the longer side and the shorter side at the leading edge at the beveled proximal end of the beveled stent mounted on the primary expandable stent delivery balloon, and
a distal radiopaque marker positioned at the distal portion of the primary expandable stent delivery balloon to identify, at the distal end of the beveled stent, the longer side of the beveled stent.

15. The method according to claim 14 wherein a hypotenuse of the triangular shaped proximal radiopaque marker demarcates the ostium of the branch vessel; and wherein the distal radiopaque marker comprises an arrow-shaped distal radiopaque marker.

16. The method according to claim 14 wherein the beveled stent is radiopaque and has a distal end and a beveled proximal end with a bevel angle at a leading edge and defined by the longer side and the shorter side of the beveled stent.

17. The method according to claim 16 wherein the bevel angle is less than 90 degrees to conform to the ostium of the branch vessel arising with less than a 90-degree angle from the main vessel.

18. The method according to claim 14 further comprising:
attaching a secondary expandable balloon, having a proximal portion and a distal portion, to an upper side of the elongated shaft of the catheter that corresponds to the longer side of the beveled stent, and proximally adjacent to the proximal portion of the primary expandable stent delivery balloon that includes the triangular shaped proximal radiopaque marker positioned thereon; and
inflating and deflating the secondary expandable balloon independently from the primary expandable stent delivery balloon via a secondary inflation lumen that extends in the catheter from the proximal end to the secondary expandable balloon and is in fluid communication therewith;
wherein the secondary expandable balloon is filled with contrast and remains outside the ostium of the branch vessel to aid in placement of the beveled stent.

19. The method according to claim 14 wherein the elongated shaft of the catheter includes an angulation proximal to the beveled stent mounted on the primary expandable stent delivery balloon; and wherein the angulation corresponds to an angle between the main vessel and the branch vessel.

20. The method according to claim 14 wherein the radially expandable beveled stent is radially expandable, via inflation of the primary expandable stent delivery balloon, from a non-deployed state to a deployed state; wherein a difference in length (ΔL) between the longer side and the shorter side of the radially expandable stent is calculated based upon the angle (Θ), defined as 90 minus a determined angle of the branch vessel, and the radius (r) of the branch vessel using an equation:
ΔL=(tangent Θ)×r.
